(19) United States
(12) Plant Patent Application Publication
(10) Pub. No.: US 2005/0210554 P1
(43) Pub. Date: Sep. 22, 2005

(54) PRUNUS ROOTSTOCK NAMED 'ELTA'

(50) PRUNUS LANNESIANA; PRUNUS ROOTSTOCK

(75) Inventor: Gennadiy Eremin, Krasnodar (RU)

Correspondence Address: THE WEBB LAW FIRM, P.C., 700 KOPPERS BUILDING, 436 SEVENTH AVENUE, PITTSBURGH, PA, 15219, US

(73) Assignee: Varieties International, LLC, Lafayette, OR (US)

(21) Appl. No.: 10/802,633

(22) Filed: Mar. 17, 2004

(51) Int. Cl. A01H 5/00

(52) U.S. Cl. PLT/183

(57) ABSTRACT

A new and distinct Prunus sp. plant used as a rootstock that exhibits desirable propagation characteristics and pink flowers.

### BOTANICAL CLASSIFICATION

Prunus sp.

### VARIETAL DENOMINATION

‘Elta’

### BACKGROUND OF THE INVENTION

The present invention comprises a new and distinct cultivar of Prunus sp. used as a rootstock known by the varietal name ‘Elta’. The new variety was discovered in the Krasnodar region of Russia around 1987. The new variety is the result of free pollination of a Prunus lannesiana plant (female parent). The purpose of breeding ‘Elta’ was to provide a productive clonal rootstock for cherry varieties. The new variety has been trial and field tested and has been found to retain its distinctive characteristics and remain true to type through successive propagations. The following characteristics distinguish ‘Elta’ from other varieties known to the breeder:

1. ‘Elta’ has big stipules;

2. ‘Elta’ is easy to propagate via green cuttings; and

3. ‘Elta’ has pink flowers in bunches.

### DESCRIPTION OF THE DRAWINGS

The accompanying photographic drawings illustrate the new variety, with the color being as nearly true as is possible with color illustrations of this type:

FIG. 1 shows a close-up view of the flowers of the new variety;

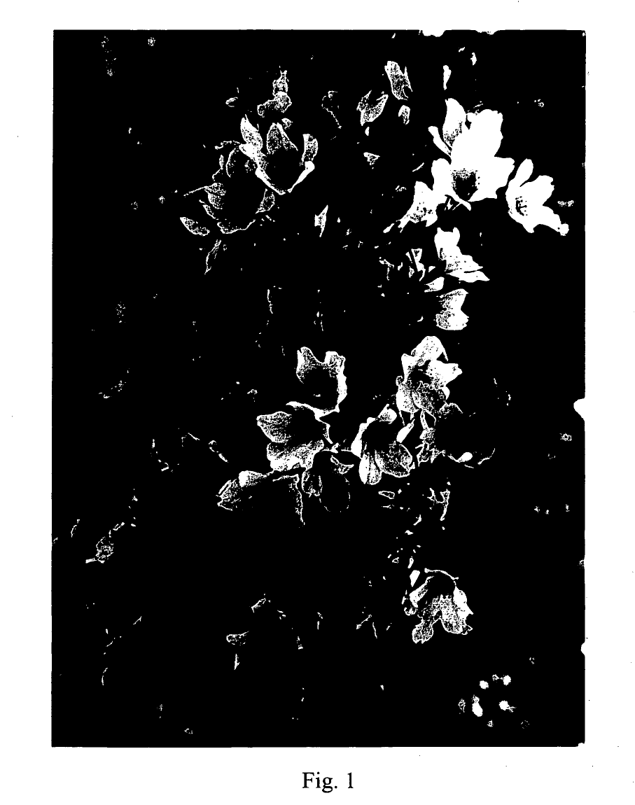

FIG. 2 shows the new variety grown to a flowering plant; and

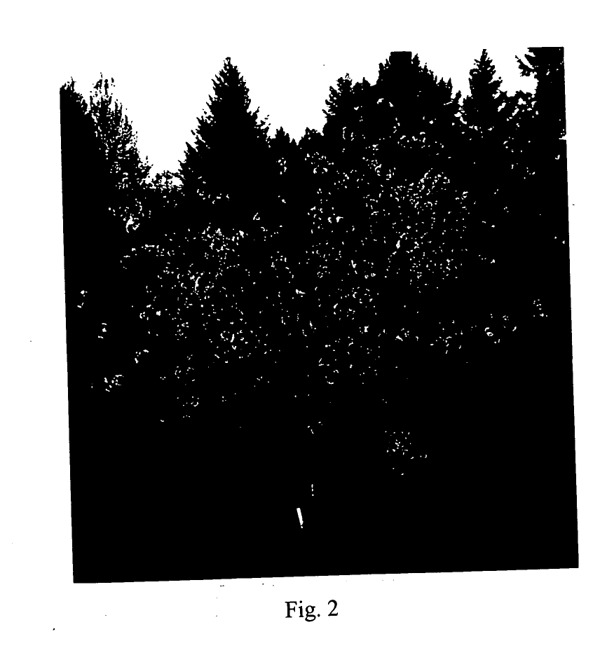

FIG. 3 illustrates the roots and branches of the new variety.

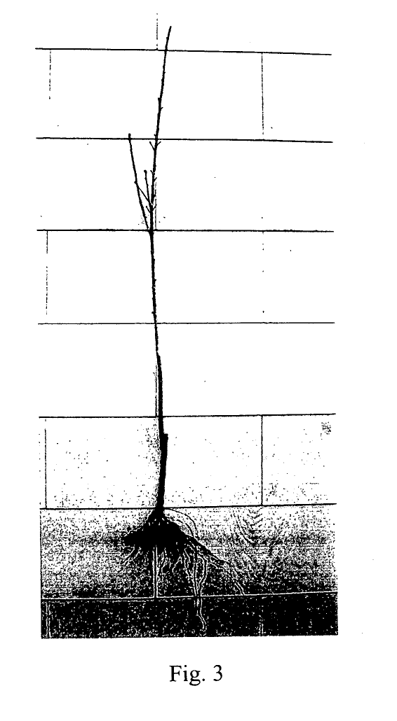

### DETAILED BOTANICAL DESCRIPTION

The following detailed description sets forth the characteristics of the new cultivar. The data which defines these characteristics were collected by asexual reproductions via green cuttings carried out in the Krasnodar Region of Russia. The new variety was grown under warm, dry Summer conditions with a temperature range of 70 to 105° F. The Winter months are mild with lows to −10° F. The Spring and Fall months are humid. The color readings were taken in natural daylight.

### Tree

Use: Rootstock.
Fruit bearing: High productivity of grafted scion.
Size (generally): Medium.
Height at 4 years: 8 feet.
Spread at 4 years: 5 feet.
Form: Rounded.
Trunk:
   Size.—Medium.
   Bark color.—187A.
   Diameter.—23.0 mm at 4 years when grown in a pot.
   Texture.—Smooth.
   Lenticel number.—6.0-15.0 per cm at 4 years.
Branches:
   Surface texture.—Smooth.
   Young bark color.—184C.
   Mature bark color.—187C.
   Internode length.—2.3-3.0 cm.
   Lenticel shape.—Round.
   Lenticel number.—2.0-5.0 per cm on a 1 year old branch.
   Lenticel size.—1 mm.
   Bud shape.—Conical.
   Bud size.—6.0 mm.
   Diameter.—5.0 mm at 1 year.
   Crotch angle.—30-550.
Leaves:
   Bud.—Length: 2.0 mm. Diameter: 1.0 mm. Color: 200D.
   Overall size.—Large.
   Length.—15.0 cm.
   Width.—6.0 cm.
   Surface texture.—Wrinkled.
   Form.—Broad; elliptic.
   Color.—Lower surface: Light grey. Upper surface Green.
   Mid-vein.—Size: Medium. Color: Light pink.
   Petiole.—Length: 2.5 cm. Thickness: 1.0-1.5 mm. Color: Pink.
   Leaf glands.—Absent.
   Stipules.—Shape: Round. Size: Up to 2.0 cm. Color: Green.
Flowers:
   Bloom time.—Flowers reach full bloom in the first week of April and last approximately 10 days.
   Size.—Large; Approximately 30.0-35.0 mm.
   Color.—Light pink.
   Number.—2 to 3 flowers per cluster.
   Fragrance.—None.
Petals:
   Number.—5 per flower.
   Length.—15.0 mm.
   Width.—Widest point is 12 mm; the base and apex are both 10 mm.
   Shape.—Ovoid.
   Margin.—Entire, with a forked split at the tip.
   Texture.—Smooth.
   Appearance.—Petals overlap.
   Color (both surfaces).—155C background with 10% of the petal having 66D at the base in a fan shape.
Sepals:
   Shape.—Triangular.
   Margin.—Entire.
   Texture.—Smooth.
   Length.—5.0 mm.
   Width.—2.0 mm.
   Color (both surfaces): 138A.
Reproductive organs:
   Stamens.—Number (per flower): 27. Filament length: 3.0 mm. Anthers: Shape: Round. Length: Less than 1.0 mm. Color: 26A. Pollen: Color: 21A. Amount: Light.
   Pistils.—Length: 4.0 mm. Style: Length: 4.0 mm. Color: 145C. Stigma: Shape: Round. Color: 144A.
Fruit:
   Size.—Small.
   Axial diameter (average).—0.8 cm.
   Axial diameter transverse in the suture plane.—0.7 cm.
   Axial diameter transverse and at a right angle to the suture plane.—0.8 cm.
   Shape.—Symmetric.
   Stem cavity.—Shape: Round. Depth: Small. Breadth: Narrow.
   Base shape.—Round.
   Apex shape.—Round.
   Skin.—Thickness: Thin. Texture: Tender. Color: Black-violet.
   Flesh.—Texture: Tender. Juice: Dark red.
   Aroma.—Absent.
   Flavor.—Bitter.
   Use.—Not recommended for eating.

### Soil Adaptation and Tolerance

Wet soil: Resilient.
Cold temperatures: Hardy to Zone 4.
Drought: Tolerant.
Asphixia: Tolerant.

### Multiplication Ability

Layering: Weak propagation.
Hardwood cuttings: Resistant to no propagation.
Softwood cuttings: Great propagation.

### Pathogen Resistance

Fungal disease: Tolerant.
Insects: Not resistant.
Mites: Weak resistance.
Viruses: Tolerant.
Diseases: Tolerant to Coccomyces and Verticillium; sensitive to Agrobacterium.
Root-knot nematodes: Susceptible; galls do not form, but nematode reproduces well.
Lesion nematodes: Moderately resistant.

### Performance as a Rootstock When Grafted

Root sprouts (suckering): Suckers (shoots that grow from the rootstock and disrupt the growth of the scion) are not present.
Anchorage: Very good.
Compatibility: Compatible with all varieties of sweet and sour cherry varieties known to the breeder.
Vigor: Medium; displaying fast growth in hot conditions.

1. A new and distinct variety of sp. plant, as illustrated and described.
